(19) United States
(12) Patent Application Publication
(10) Pub. No.: US 2009/0123366 A1
(43) Pub. Date: May 14, 2009

(54) Targeted Therapy

(75) Inventors: Jon Dobson, Stoke-On-Trent (GB); Claire Lewis, Sheffield (GB); Helen Byrne, Sheffield (GB)

Correspondence Address: SWANSON & BRATSCHUN, L.L.C., 8210 SOUTHPARK TERRACE, LITTLETON, CO, 80120, US

(73) Assignee: Keele University, Staffordshire (GB)

(21) Appl. No.: 12/293,452

(22) PCT Filed: Apr. 3, 2007
(86) PCT No.: PCT/GB07/01272

§ 371 Date: Jan. 13, 2009

Related U.S. Application Data
(60) Provisional application No. 60/789,185, filed on Apr. 3, 2006.

(30) Foreign Application Priority Data
Apr. 3, 2006 (GB) 0606660.9

(51) Int. Cl. A61K 51/12; C12N 5/06; A61K 39/395; A61K 35/12; A61K 31/711; A61K 9/00

(52) U.S. Cl. 424/1.17; 435/325; 424/400; 424/130.1; 514/44; 435/375; 424/93.71

(57) ABSTRACT

In order to target a diseased material in a subject, a monocyte, or monocyte-derived cell, such as a macrophage, which incorporates a magnetic material, such as a magnetic particle or a ferrofluid, preferably having a biocompatible coating, is proposed to be administered. A magnetic energy source may then be applied to the subject to destroy, rupture or inactivate the diseased material. Alternatively, the monocyte or monocyte derived cell may additionally include a therapeutic agent, which is thereby targeted at the diseased material.

### FIELD OF THE INVENTION

The present invention relates to cells, in particular macrophages, containing a magnetic material and their use in the treatment of tumours.

### BACKGROUND TO THE INVENTION

In recent years, important new insights into the molecular mechanisms underpinning the growth and spread of malignant tumours have emerged. This has prompted the design of a number of therapeutic agents that specifically inhibit these aberrant cellular processes. These usually take the form of a peptide/protein (or a low molecular weight mimetic) which binds to either an extracellular protein to inhibit its activity (e.g. an enzyme such as a matrix metalloproteinase) or a molecule in or on the target cell to modify its activity (e.g. the function of tyrosine kinases in the membrane). However, attempts to target and limit the delivery of such drugs to the tumour site have proved largely unsuccessful, with many showing only limited penetration into the tumour and prominent side effects in clinical trials due to their adverse effects on non-malignant tissues. Overcoming this problem of targeted delivery is thought to be crucial for the long-term and effective administration of such agents.

More recently, various forms of cancer gene therapy are attempting to transfer an anti-tumour gene to target cells in tumours (e.g. tumour cells, or endothelial cells in blood vessels). The likelihood of such an approach playing an effective role in cancer treatment has been boosted by the recent characterisation of the human genome and improvements in gene transfer technology. Indeed, more than 150 clinical trials of such anti-cancer gene therapies are in progress. Strategies include replacing mutated or deleted tumour suppressor genes with their wild type counterparts, suppression of the expression/activity of oncogenes (or their downstream effectors), and the delivery of genes encoding cytotoxic proteins, pro-drug activating enzymes, immuno-modulatory or anti-angiogenic proteins to the tumour site (reviewed in Kouraklis, 2000). Although a wide range of viral and non-viral vectors has been used to deliver such genes to tumours, these have met with limited success; the major obstacle being the development of a tumour-specific delivery system (Vile et al. 2000). Accordingly, many anti-cancer gene therapy protocols involve local administration of vectors (e.g. by needle injection directly into the primary tumour or its blood supply). This has limited applicability to patients with disseminated disease because metastatic tumours are often undetected, too numerous and/or inaccessible to direct injection. An alternative approach has been to try to deliver vectors systemically (so they are carried into both primary and metastatic tumours), but to restrict gene expression to the tumour site by placing the therapeutic gene under the control of a promoter (or part thereof) responsive to a tumour-specific condition (Brown, 2000). One such signal may be hypoxia (low oxygen).

Oxygen microelectrodes have been used extensively to measure oxygen levels in human tumours. These studies have demonstrated the presence of many areas of hypoxia (low oxygen) and anoxia (no oxygen) in different tumour types, including those of the brain, breast, cervix, head/neck and soft tissue sarcomas (Vaupel et al. 1989; Brown, 2000). Whereas normal tissues typically have median oxygen tensions of 30-70 mmHg, over half of all solid tumours examined exhibited median values of <10 mm Hg (with fewer than 10% of measurements in the normal range). These hypoxic/anoxic regions appear because the newly formed blood vessels in tumours are often disorganised with many blind ends, incomplete endothelial linings and basement membranes, and have a tendency to collapse (Brown & Giaccia, 1998). Consequently blood flow is sluggish and irregular and the delivery of oxygen and nutrients is poor to many regions of the tumour. The rapid expansion and oxygen consumption of tumour cells around these new blood vessels also contributes to the level of hypoxia formed, although once a threshold level of hypoxia is reached, tumour cells in that area stop proliferating and switch to anaerobic glycolysis for energy production (reviewed by Brown, 2000).

Various studies have shown that the presence of large areas of hypoxia in tumours is correlated with poor prognosis. This is thought to be because hypoxic tumour cells are relatively resistant to such conventional anti-cancer therapies as radiotherapy and chemotherapy. Well-oxygenated tumour cells are markedly more responsive to radiotherapy than their hypoxic counterparts because oxygen-derived free radicals potentiate the protein and DNA damage induced by the ionising radiation. Most anti-cancer chemotherapeutic agents only kill tumour cells if they are rapidly proliferating, so the non-proliferative hypoxic fractions of tumours are relatively resistant to their effects. While in this non-proliferative state, hypoxic tumour cells are also known to secrete cytokines and enzymes to induce the growth of new blood vessels within the tumour, thereby providing oxygen and nutrients for tumour growth as well as increased exit routes for tumour cells into the general circulation. Hypoxia also exerts a selective pressure on tumour cells because only those with an aggressive phenotype (e.g. mutated for the tumour suppressor gene, p53) are able to survive hypoxia, and go on to re-populate the tumour and metastasise to distant sites (Brown & Giaccia, 1998; Brown, 2000). As these hypoxic areas are relatively inaccessible to conventional anti-cancer drugs and gene vectors (due to the absence of a blood supply) there is a need for therapies that are capable of penetrating these regions in tumours.

Recent studies showed that monocytes, which enter human tumours at a constant rate from the bloodstream and then differentiate into macrophages, rapidly accumulate in such hypoxic/necrotic areas (Leek et al., 1996; Burton et al., 2001, Burke et al 2003). The idea that macrophages might be useful as delivery vehicles to target gene therapy to hypoxic tumour sites was investigated. In this approach, autologous macrophages are ‘armed’ with a therapeutic gene whose expression is induced by hypoxia (Griffiths et al., 2000). So, macrophages would be taken from the bloodstream of a given cancer patient, differentiated into macrophages ex vivo, transfected with a hypoxia-activated therapeutic gene, and then re-infused into the patient. The transfected macrophages are then taken up from the bloodstream into the primary tumour (as well as any secondary tumours present elsewhere in the body) and accumulate in hypoxic tumour areas. When macrophages transfected with a hypoxia-regulated gene (eg. a prodrug-activating enzyme such as cytochrome, P450), were co-cultured in vitro with breast tumour multicell spheroids (small 3-D tumour masses grown in vitro from breast tumour cell lines, they rapidly migrated into the inner, hypoxic regions of these small tumour masses and expressed the transgene. When MTS were then exposed to the prodrug, cyclophosphamide, the P450 enzyme expressed by hypoxic macrophages at the centre of the MTS converted the prodrug into its active, cytotoxic metabolite. This then diffused out of the macrophages (which are non-dividing cells and thus refractory to its effects), was taken up by tumour cells and intercalated into their DNA, causing cell death during their subsequent mitosis (Griffiths et al., 2000).

However, there has been little evidence to date showing that macrophages manipulated ex vivo are then capable of trafficking to the tumour site in large numbers when re-injected into tumour-bearing mice. Indeed, macrophages stimulated to be cytotoxic towards tumour cells in vitro (eg. by exposure to cytokines ex vivo) rapidly became trapped in the fenestrated capillaries of the liver, lungs and kidneys following re-infusion into tumour bearing mice (Bartholeyns et al 194 & 1996) or cancer patients (Andreesen et al 1998).

In view of the above, there is a need to target macrophages into diseased material, for example a tumour.

### STATEMENTS OF INVENTION

According to a first aspect of the invention there is provided a monocyte, or monocyte-derived cell, comprising a magnetic material.

The monocyte, or monocyte derived cell, of the invention, referred to hereinafter as “cell”, is useful as a vehicle for targeting a therapeutic agent to a diseased material. Moreover the cell of the invention is useful in the thermal treatment, for example, hyperthermia, of a tumour.

The monocyte derived cell is preferably a macrophage. Macrophages according to the invention are useful in the treatment of diseased material such as a tumour because (i) able to easily penetrate the endothelial walls (ii) naturally migrate into, and accumulate in, poorly vascularised areas, for example in a tumour and (iii) avoid the possibility of embolisation of the blood vessels in the target region due to accumulation of the magnetic material since this will be inside a cellular carrier i.e. the macrophage.

The expression “magnetic material” is used herein to refer to a material that when exposed to a magnetic field either heats or physically moves. Preferably the magnetic material takes the form of a magnetic particle, for example a micro- or nano-particle.

Alternatively the magnetic material may be a fluid, for example, a fluid in which magnetic particles are in suspension otherwise known as a ferrofluid.

The magnetic particles will generally be spherical or elliptical and have a mean size in the range 1 nm to 10 μm. The particles may have a mean size of between 5 nm and 10 μm, for example between 10 μm and 1 μm. Preferably the particles are nano-particles having a mean size or diameter of, for example, 5000 nm or less, e.g. from 1 nm to 5000 nm, preferably from 1 nm to 1000 nm, more preferably from 1 nm to 300 nm, or from 2 nm to 10 nm.

The magnetic material may be inherently magnetic or, alternatively, may be one which reacts in a magnetic field. The magnetic material may be ferromagnetic, antiferromagnetic, ferrimagnetic, antiferrimagnetic or superparamagnetic. The magnetic material may include elemental iron, chromium manganese, cobalt, nickel, or a compound thereof. The iron compound may be an iron salt which may be selected from the group which includes magnetite (Fe₃O₄), maghemite (γFe₂O₃) and greigite (Fe₃S₄), or any combination thereof. The chromium compound may be chromium dioxide.

The magnetic material may comprise a biocompatible coating. The biocompatible coating may be a metal, for example gold, a synthetic material or a biological material or a combination thereof. The synthetic coating may be a polymer, copolymer or combination thereof. The polymer may include dextran, polyvinyl alcohol (PVA), polyethylenimine (PEI), or silica. The biocompatible coating may be provided with a biological molecule which may function to facilitate adhesion of the magnetic particle to the monocyte, or monocyte derived cell, of the invention. Examples of molecules that can be used to facilitate adhesion include RGD (synthetic peptide containing the arginine-glycine-aspartate sequence motif), transferrin, collagen, fibronectin/fibrin, ion channel receptors, cell specific integrin receptors or any cell surface antigen.

The cell according to the invention may comprise a detectable agent and/or a therapeutic agent. The detectable agent may be a detectable label which may be linked to a therapeutic agent. Preferably the detectable agent can be visualised by MRI.

In a preferred aspect of the invention cell, preferably a macrophage, of the invention comprises a therapeutic agent. As used herein the term “therapeutic agent” is intended to include any agent useful in therapy. The therapeutic agent may include a chemotherapeutic agent, a radiotherapeutic agent, or a gene, or part thereof, (useful in gene therapy).

A chemotherapeutic agent may be an agent selected from the group consisting of S phase dependent antimetabolics, capercitabine, cytarabine, doxorubicin, fludarabine, floxuridine, fluorouracil, gemcitabine, hydroxyurea, mercaptopurine, methotrexate, prednisone, procarbazine, thioguanine, M phase dependent vinca alkaloids, vinblastine, vincristine, vinorelbine, podophyllotoxins, etoposide, teniposide, taxanes, doxetaxel, paxlitaxel, G2 phase dependent, bleomycin, irinotecan, mitoxantrone, topotecan, G1 phase dependent, asparaginase, corticosteroids, alkylating agents, nitrogen mustards, mechlorethamine, mustargen, cyclophosphamide, ifosfamide and clorambucil, leukeran, nitrosoureas, platinum agents, cisplatin, platinol, carboplatin, paraplatin, antimetabolites, natural therapeutic products, antitumour antibiotics, anthracyclines, epipodophyllotoxins, vinca alkaloids, taxanes, camptothecin, melphalan, carmusline, methotrexate, 5-fluorouracil, mercaptopurine; daunorubicin; doxorubicin; epirubicin; vinblastine; vincristine; dactinomycin; mitomycin C; taxol; L-asparaginase; G-CSF; etoposide; colchicine; derferoxamine mesylate or a combination thereof.

The radiotherapeutic agent may comprise a radionuclide selected from the group consisting of Molybdenum-99, Technetium-99m, Chromium-51, Copper-64, Dysprosium-165, Ytterbium-169, Indium-111, Iodine-125, Iodine-131, Irdium-192, Iron-59, Phosphorous-32, Potassium-42, Rhodium-186, Rhenium-188, Samarium-153, Selenium-75, Sodium-24, Strontium-89, Xenon-133, Xenon-127 and Yttrium-90 or a combination thereof.

The chemotherapeutic or radiotherapeutic agent may be associated with an antibody, for example a monoclonal antibody.

The therapeutic agent may include DNA, RNA, interfering RNA (RNAi), a peptide, polypeptide, an antibody for example a monoclonal antibody or an antibody fragment such as a single chain antibody fragment, an aptamer, a small molecule. Small molecules may include, but are not limited to, peptides, peptidomimetics (e.g. peptoids), amino acids, amino acid analogs, polynucleotides, polynucleotide analogs, nucleotides, nucleotide analogs, organic or inorganic compounds (i.e. including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

Preferably the therapeutic agent is a gene, or a part thereof (referred to hereinafter as a “therapeutic gene”).

In a further preferred aspect of the invention the cell of the invention further comprises a responsive element. As used herein the term “responsive element” (otherwise known as a response element) is intended to include a gene, or part thereof, the expression or activity of which is regulated by, or responsive to, conditions specific to the diseased material to be treated. As used herein “a part of a gene” may include a regulatable element of a gene for example an enhancer, promoter or part thereof. The responsive element may be operably linked to a therapeutic gene so as to regulate expression of the therapeutic gene. Preferably the responsive element comprises all or part of a therapeutic gene. Preferably still the responsive element is a therapeutic gene or part thereof.

Responsive elements in accordance with the invention include hypoxia inducible elements (“hypoxia response elements”) and anoxia inducible elements (“anoxia response elements”) examples of which are known in the art and include, but are not limited to, hypoxia inducible factor-(HIF-1), activating transcription factor 4 (ATF-4) and the transcription factor, VL30. Hypoxia inducible, or regulated, elements are activated by low levels of oxygen, for example between 1% and 8% oxygen. Anoxia inducible, or regulated, elements are activated by extremely low levels of oxygen for example less than 0.1% oxygen.

The responsive element may be specific to a tumour (hereinafter referred to as a “tumour responsive element”) such that the activity or expression of the tumour responsive element is regulated by, or responsive to, conditions in or around a tumour, for example hypoxia, anoxia, low pH (acidic), high pH (basic), hypoglycaemia. Preferably the responsive element is hypoxia or anoxia regulated such as described herein.

A therapeutic gene, or responsive element, in accordance with the invention may include, but is not limited to, a tumour suppressor gene, antigenic gene, cytotoxic gene, cytostatic gene, cytokine gene, chemokine gene, pharmaceutical protein gene, pro-apoptotic gene, pro-drug activating gene or an anti-angiogenic gene.

The term “tumor suppressor gene” refers to a nucleotide sequence, the expression of which in the target cell is capable of suppressing the neoplastic phenotype and/or inducing apoptosis. Examples of tumor suppressor genes useful in the practice of the present invention include the p53 gene, the APC gene, the DPC-4 gene, the BRCA-1 gene, the BRCA-2 gene, the WT-1 gene, the retinoblastoma gene (Lee, et al. (1987) Nature 329:642), the MMAC-1 gene, the adenomatous polyposis coli protein (Albertsen, et al., U.S. Pat. No. 5,783,666 issued Jul. 21, 1998), the deleted in colon carcinoma (DCC) gene, the MMSC-2 gene, the NF-1 gene, nasopharyngeal carcinoma tumor suppressor gene that maps at chromosome 3p21.3. (Cheng, et al. 1998. Proc. Nat. Acad. Sci. 95:3042-3047), the MTS1 gene, the CDK4 gene, the NF-1 gene, the NF2 gene, and the VHL gene. A particularly preferred adenovirus for therapeutic use is the A/C/N/53 vector encoding the p53 tumor suppressor gene as more fully described in Gregory, et al., U.S. Pat. No. 5,932,210 issued Aug. 3, 1999, the entire teaching of which is herein incorporated by reference.

The term “antigenic genes” refers to a nucleotide sequence, the expression of which in the target cells results in the production of a cell surface antigenic protein capable of recognition by the immune system. Examples of antigenic genes include carcinoembryonic antigen (CEA), p53 (as described in Levine, A. PCT International Publication No. WO94/02167 published Feb. 3, 1994). In order to facilitate immune recognition, the antigenic gene may be fused to the MHC class I antigen. Preferably the antigenic gene is derived from a tumour cell specific antigen. Ideally a tumour rejection antigen. Tumour rejection antigens are well known in the art and include, by example and not by way of limitation, the MAGE, BAGE, GAGE and DAGE families of tumour rejection antigens, see Schulz et al Proc Natl Acad Sci USA, 1991, 88, pp 991-993.

It has been known for many years that tumour cells produce a number of tumour cell specific antigens, some of which are presented at the tumour cell surface. These are generally referred to as tumour rejection antigens and are derived from larger polypeptides referred to as tumour rejection antigen precursors. Tumour rejection antigens are presented via HLA's to the immune system. The immune system recognises these molecules as foreign and naturally selects and destroys cells expressing these antigens. If a transformed cell escapes detection and becomes established a tumour develops. Vaccines have been developed based on dominant tumour rejection antigen's to provide individuals with a preformed defence to the establishment of a tumour.

The term “cytotoxic gene” refers to nucleotide sequence, the expression of which in a cell produces a toxic effect. Examples of such cytotoxic genes include nucleotide sequences encoding pseudomonas exotoxin, ricin toxin, diptheria toxin, and the like.

The term “cytostatic gene” refers to nucleotide sequence, the expression of which in a cell produces an arrest in the cell cycle. Examples of such cytostatic genes include p21, the retinoblastoma gene, the E2F-Rb gene, genes encoding cyclin dependent kinase inhibitors such as P16, p15, p18 and p19, the growth arrest specific homeobox (GAX) gene as described in Branellec, et al. (PCT Publication WO97/16459 published May 9, 1997 and PCT Publication WO96/30385 published Oct. 3, 1996).

The term “cytokine gene” refers to a nucleotide sequence, the expression of which in a cell produces a cytokine. Examples of such cytokines include GM-CSF, the interleukins, especially IL-1, IL-2, IL-4, IL-12, IL-10, IL-19, IL-20, interferons of the α, β and γ subtypes, consensus interferons and especially interferon α-2b and fusions such as interferon α-2α-1.

The term “chemokine gene” refers to a nucleotide sequence, the expression of which in a cell produces a cytokine. The term chemokine refers to a group of structurally related low-molecular cytokines weight factors secreted by cells are structurally related having mitogenic, chemotactic or inflammatory activities. They are primarily cationic proteins of 70 to 100 amino acid residues that share four conserved cysteine. These proteins can be sorted into two groups based on the spacing of the two amino-terminal cysteines. In the first group, the two cysteines are separated by a single residue (C-x-C), while in the second group, they are adjacent (C—C). Examples of member of the ‘C-x-C’ chemokines include but are not limited to platelet factor 4 (PF4), platelet basic protein (PBP), interleukin-8 (IL-8), melanoma growth stimulatory activity protein (MGSA), macrophage inflammatory protein 2 (MIP-2), mouse Mig (m119), chicken 9E3 (or pCEF-4), pig alveolar macrophage chemotactic factors I and II (AMCF-I and -II), pre-B cell growth stimulating factor (PBSF),and IP10. Examples of members of the ‘C—C’ group include but are not limited to monocyte chemotactic protein 1 (MCP-1), monocyte chemotactic protein 2 (MCP-2), monocyte chemotactic protein 3 (MCP-3), monocyte chemotactic protein 4 (MCP-4), macrophage inflammatory protein 1 α (MIP-1-α), macrophage inflammatory protein 1 β (MIP-1-β), macrophage inflammatory protein 1-γ (MIP-1-γ), macrophage inflammatory protein 3 α (MIP-3-α), macrophage inflammatory protein 3 β (MIP-3-β), chemokine (ELC), macrophage inflammatory protein-4 (MIP-4), macrophage inflammatory protein 5 (MIP-5), LD78 β, RANTES, SIS-epsilon (p500), thymus and activation-regulated chemokine (TARC), eotaxin, I-309, human protein HCC-1/NCC-2, human protein HCC-3, mouse protein C10.

The term “pharmaceutical protein gene” refers to nucleotide sequence, the expression of which results in the production of protein have pharmaceutically effect in the target cell. Examples of such pharmaceutical genes include the proinsulin gene and analogs (as described in PCT International Patent Application No. WO98/31397, growth hormone gene, dopamine, serotonin, epidermal growth factor, GABA, ACTH, NGF, VEGF (to increase blood perfusion to target tissue, induce angiogenesis, PCT publication WO98/32859 published Jul. 30, 1998), thrombospondin etc. Also, the pharmaceutical protein gene may encompass immunoreactive proteins such as antibodies, Fab fragments, Fv fragments, humanized antibodies, chimeric antibodies, single chain antibodies, and human antibodies derived from non-human sources.

The term “pro-apoptotic gene” refers to a nucleotide sequence, the expression thereof results in the induction of the programmed cell death pathway of the cell. Examples of pro-apoptotic genes include p53, adenovirus E3-11.6K(10.5K), the adenovirus E4orf4 gene, p53 pathway genes, and genes encoding the caspases.

The term “pro-drug” is intended to any non-therapeutic compound which is a latent therapeutic compound that is capable of being converted to a therapeutic compound. The term “pro-drug activating genes” refers to nucleotide sequences, the expression of which, results in the production of protein capable of converting a non-therapeutic compound (i.e. a pro-drug) into a therapeutic compound (i.e. a drug) which renders the cell susceptible to killing by external factors or causes a toxic condition in the cell. An example of a prodrug activating gene is the cytosine deaminase gene. Cytosine deaminase converts 5-fluorocytosine to 5 fluorouracil, a potent antitumor agent). The lysis of the tumor cell provides a localized burst of cytosine deaminase capable of converting 5FC to 5FU at the localized point of the tumor resulting in the killing of many surrounding tumor cells. This results in the killing of a large number of tumor cells without the necessity of infecting these cells with an adenovirus (the so-called bystander effect”). Additionally, the thymidine kinase (TK) gene (see e.g. Woo, et al. U.S. Pat. No. 5,631,236 issued May 20, 1997 and Freeman, et al. U.S. Pat. No. 5,601,818 issued Feb. 11, 1997) in which the cells expressing the TK gene product are susceptible to selective killing by the administration of gancyclovir may be employed. The pro-drug activating gene may be the hypoxia regulated gene, cytochrome p450.

The term “anti-angiogenic” genes refers to a nucleotide sequence, the expression of which results in the extracellular secretion of anti-angiogenic factors. Anti-angiogenesis factors include angiostatin, inhibitors of vascular endothelial growth factor (VEGF) such as Tie 2 (as described in PNAS (USA) (1998) 95:8795-8800), endostatin.

It will be readily apparent to those of skill in the art that modifications and or deletions to the above referenced genes so as to encode functional subfragments of the wild type protein may be readily adapted for use in the practice of the present invention. For example, the reference to the p53 gene includes not only the wild type protein but also modified p53 proteins. Examples of such modified p53 proteins include modifications to p53 to increase nuclear retention, deletions such as the □13-19 amino acids to eliminate the calpain consensus cleavage site (Kubbutat and Vousden (1997) Mol. Cell. Biol. 17:460-468, modifications to the oligomerization domains (as described in Bracco, et al. PCT published application WO97/0492 or U.S. Pat. No. 5,573,925, etc.).

It will be readily apparent to those of skill in the art that the above therapeutic genes may be secreted into the media or localized to particular intracellular locations by inclusion of a targeting moiety such as a signal peptide or nuclear localization signal (NLS). Also included in the definition of therapeutic transgene are fusion proteins of the therapeutic transgene with the herpes simplex virus type 1 (HSV-1) structural protein, VP22. Fusion proteins containing the VP22 signal, when synthesized in an infected cell, are exported out of the infected cell and efficiently enter surrounding non-infected cells to a diameter of approximately 16 cells wide. This system is particularly useful in conjunction with transcriptionally active proteins (e.g. p53) as the fusion proteins are efficiently transported to the nuclei of the surrounding cells. See, e.g. Elliott, G. & O'Hare, P. Cell. 88:223-233:1997; Marshall, A. & Castellino, A. Research News Briefs. Nature Biotechnology. 15:205:1997; O'Hare, et al. PCT publication WO97/05265 published Feb. 13, 1997. A similar targeting moiety derived from the HIV Tat protein is also described in Vives, et Al. (1997) J. Biol. Chem. 272:16010-16017.

In a further aspect of the invention there is provided a cell, such as a macrophage, according to the invention for use as a medicament.

In a further aspect of the invention there is provided a pharmaceutical composition comprising a cell, preferably a macrophage, according to the invention. When administered, the pharmaceutical compositions of the present invention are administered in pharmaceutically acceptable preparations. Such preparations may routinely contain pharmaceutically acceptable concentrations of salt, buffering agents, preservatives, compatible carriers, supplementary immune potentiating agents such as adjuvants and cytokines and optionally other therapeutic agents for example a chemotherapeutic agent.

The therapeutics of the invention can be administered by any conventional route, including injection or by gradual infusion over time. The administration may, for example, be oral, intravenous, intraperitoneal, intramuscular, intracavity, subcutaneous, or transdermal.

The compositions of the invention are administered in effective amounts. An “effective amount” is that amount of a composition that alone, or together with further doses, produces the desired response. In the case of treating cancer, the desired response is inhibiting the progression of the disease. This may involve only slowing the progression of the disease temporarily, although more preferably, it involves halting the progression of the disease permanently. This can be monitored by routine methods or can be monitored according to diagnostic methods of the invention discussed herein.

Such amounts will depend, of course, on the particular condition being treated, the severity of the condition, the individual patient parameters including age, physical condition, size and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

The pharmaceutical compositions used in the foregoing methods preferably are sterile and contain an effective amount of nucleic acid for producing the desired response in a unit of weight or volume suitable for administration to a patient. The response can, for example, be measured by determining regression of a tumour, decrease of disease symptoms, modulation of apoptosis, etc.

The doses of therapeutic agent administered to a subject can be chosen in accordance with different parameters, in particular in accordance with the mode of administration used and the state of the subject. Other factors include the desired period of treatment. In the event that a response in a subject is insufficient at the initial doses applied, higher doses (or effectively higher doses by a different, more localized delivery route) may be employed to the extent that patient tolerance permits.

In general, doses of therapeutic agent of between 1 nM-1 μM generally will be formulated and administered according to standard procedures. Preferably doses can range from 1 nM-500 nM, 5 nM-200 nM, 10 nM-100 nM. Other protocols for the administration of compositions will be known to one of ordinary skill in the art, in which the dose amount, schedule of injections, sites of injections, mode of administration (e.g., intra-tumour) and the like vary from the foregoing. Administration of compositions to mammals other than humans (e.g. for testing purposes or veterinary therapeutic purposes) is carried out under substantially the same conditions as described above. A subject, as used herein, is a mammal, preferably a human, and including a non-human primate, cow, horse, pig, sheep, goat, dog, cat or rodent.

When administered, the pharmaceutical preparations of the invention are applied in pharmaceutically-acceptable amounts and in pharmaceutically-acceptable compositions. The term “pharmaceutically acceptable” means a non-toxic material that does not interfere with the effectiveness of the biological activity of the active ingredients. Such preparations may routinely contain salts, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

Compositions may be combined, if desired, with a pharmaceutically-acceptable carrier. The term “pharmaceutically-acceptable carrier” as used herein means one or more compatible solid or liquid fillers, diluents or encapsulating substances which are suitable for administration into a human. The term “carrier” denotes an organic or inorganic ingredient, natural or synthetic, with which the active ingredient is combined to facilitate the application. The components of the pharmaceutical compositions also are capable of being co-mingled with the molecules of the present invention, and with each other, in a maimer such that there is no interaction which would substantially impair the desired pharmaceutical efficacy.

The pharmaceutical compositions may contain suitable buffering agents, including: acetic acid in a salt; citric acid in a salt; boric acid in a salt; and phosphoric acid in a salt.

The pharmaceutical compositions also may contain, optionally, suitable preservatives, such as: benzalkonium chloride; chlorobutanol; parabens and thimerosal.

The pharmaceutical compositions may conveniently be presented in unit dosage form and may be prepared by any of the methods well-known in the art of pharmacy. All methods include the step of bringing the active agent into association with a carrier which constitutes one or more accessory ingredients. In general, the compositions are prepared by uniformly and intimately bringing the active compound into association with a liquid carrier, a finely divided solid carrier, or both, and then, if necessary, shaping the product.

Compositions suitable for oral administration may be presented as discrete units, such as capsules, tablets, lozenges, each containing a predetermined amount of the active compound. Other compositions include suspensions in aqueous liquids or non-aqueous liquids such as syrup, elixir or an emulsion.

Compositions suitable for parenteral administration conveniently comprise a sterile aqueous or non-aqueous preparation of therapeutic agent, which is preferably isotonic with the blood of the recipient. This preparation may be formulated according to known methods using suitable dispersing or wetting agents and suspending agents. The sterile injectable preparation also may be a sterile injectable solution or suspension in a non-toxic parenterally-acceptable diluent or solvent, for example, as a solution in 1,3-butane diol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono-or di-glycerides. In addition, fatty acids such as oleic acid may be used in the preparation of injectables. Carrier formulation suitable for oral, subcutaneous, intravenous, intramuscular, etc. administrations can be found in Remington's Pharmaceutical Sciences, Mack Publishing Co., Easton, Pa.

A further aspect of the invention provides a magnetic material composition, the composition comprising a macrophage comprising a magnetic material associated with a first therapy, and a therapeutic gene associated with a second therapy. The first therapy may be thermotherapy, for example hyperthermia. The second therapy may be gene therapy. The composition may also comprise at least one other agent, for example a pro-drug, associated with the therapeutic gene.

A further aspect of the invention provides a method of preparing a cell according to the invention, the method comprising introducing the magnetic material into the monocyte or monocyte derived cell. Preferably the cell is a macrophage and the magnetic material is accumulated by the macrophage during culture of the macrophage with the magnetic material.

In a preferred aspect the method includes the step of introducing the therapeutic agent into the cell according to the invention. Where the therapeutic agent is a therapeutic gene, the cell, typically a macrophage, is transfected with the gene, using a gene transfer vector. Such vectors may include liposomes and viral vectors. Viral vectors may include adenovirus, retrovirus, adeno-associated virus and sendai virus. High level gene transfer to macrophages may be achieved using such replication-deficient adenoviruses as adv5

In a further aspect of the invention there is provided a method for targeting a cell, preferably a macrophage, according to the invention to a diseased material in a subject the method comprising administering the monocyte, or monocyte derived cell, to the subject's body, body part, tissue or body fluid and exposing the monocyte, or monocyte derived cell, to a magnetic field. The administration of a magnetic field source serves to aid the macrophages natural homing ability in directing the macrophage to the diseased target. In this way, the macrophage selves to target the magnetic material to, and locating the magnetic material in, the diseased material. Upon exposure to time-varying magnetic fields, the magnetic material couples to the field and is heated.

As used herein the expression “diseased material” is intended to include diseased, disease-causing (such as pathogens implicated in disease) or undesirable material in the body or body part of a subject.

In a further aspect of the invention there is provided a method of targeting a magnetic material to a diseased material in a subject, the method comprising the administration of a cell according to the invention to the subject's body, body part, tissue or body fluid only. The method of the invention is useful in targeted thermotherapy whereby the administration of a magnetic field, preferably a time-varying magnetic field, serves to excite/heat the magnetic material of cell of the invention resulting in the release of thermal energy.

The methods of the invention may be useful in the in vivo or ex vivo targeting of the magnetic material containing cells to a diseased material in a subject.

A further aspect of the invention provides a targeted thernotherapy system for treating diseased material in a subject, the system comprising:
i) a cell, preferably a macrophage, according to the first aspect of the invention; and
ii) a magnetic energy source to heat the magnetic material within the cell.

A further aspect of the invention provides a method of treating a diseased material in a subject the method comprising the administration of a cell according to the invention, preferably a macrophage, to a subject's body, body part, tissue or body fluid and the administration of a magnetic energy source to destroy, rupture or inactivate the diseased material. Preferably the method of the invention provides for the targeted generation of heat to/at the diseased material. Such heat therapy is referred to as thennotherapy or hyperthermia. With hyperthermia, temperatures in a range of from about 40° C. to about 46° C. cause irreversible damage to cells because it induces necrosis (typically called “thermo-ablation”) and/or heat-shock response in cells (classical hyperthermia-apoptosis) leading to cell death.

In a further aspect of the invention there is provided a method of treating a diseased material in a subject the method comprising the administration of a cell according to the invention, preferably a macrophage, to a subject's body, body part, tissue or body fluid. Preferably the method is gene therapy and the cell comprises a therapeutic gene such that the gene is administered to the subject's body, body part, tissue or body fluid. The method of gene therapy may include the administration of at least one other agent, for example a pro-drug, associated with the therapeutic gene. The at least one other agent may be administered prior to, during or after the administration of the therapeutic gene.

Macrophages have been shown to migrate into the hypoxic regions of tumours. Furthermore, by the incorporation of magnetic material into the macrophages, the macrophages are magnetically targeted to these regions of the tumour. Where the macrophages comprise a hypoxia-regulated therapeutic gene, the therapeutic gene will be expressed in the hypoxic regions of the tumour.

The present invention also pertains to a method of treating a diseased material in a subject using a combination of targeted thermotherapy and gene therapy. The targeted thermotherapy may be administered to a subject before, during, after the gene therapy, or a combination thereof.

Preferably the subject is human.

The magnetic energy source is typically a magnetic field. The magnetic field may be provided by a magnet or array of magnets. The magnet or array may be a permanent magnet or an electromagnet. The magnetic field may be static or alternating, for example oscillating. Static magnetic fields are typically used when targeting the magnetic material to a diseased target; alternating magnetic fields are typically used when heating the magnetic material. The magnetic field may be administered directly into the subject's body, body part, tissue or body fluid (such as blood, blood plasma, blood serum or bone marrow), or extracorporeally to the subject's body, organ or body fluid. Preferably the magnetic field is applied externally to the body, for example over a tumour site, so as to tend to move the magnetic material (e.g. magnetic particles) in a direction towards the tumour or to a site within the tumour. In this way the magnetic field is used to target and enhance the uptake of the magnetic material containing macrophages into the tumour. The magnetic field may be applied in vivo, for example, the magnetic field may be provided by an implantable magnet implanted in the subject's body. The macrophages are capable of destroying the tumour, and macrophage itself, via magnetic particle hyperthermia.

In a further aspect of the invention there is provided the use of a cell, preferably a macrophage, according to the invention in the manufacture of a medicament for the treatment of a diseased material.

The therapeutic methods and uses of the invention may be useful in the treatment of cancer, AIDS, adverse angiogenesis, cardiovascular plaque, vascular plaque, calcified plaque, restenosis, amyloidosis, tuberculosis, obesity, arthritis.

Preferably the diseased material to be treated is cancer. As used herein, the term “cancer” refers to cells having the capacity for autonomous growth, i.e., an abnormal state or condition characterized by rapidly proliferating cell growth. The term is meant to include all types of cancerous growths or oncogenic processes, metastatic tissues or malignantly transformed cells, tissues, or organs, irrespective of histopathologic type or stage of invasiveness. The term “cancer” includes malignancies of the various organ systems, such as those affecting, for example, lung, breast, thyroid, lymphoid, gastrointestinal, and genito-urinary tract, as well as adenocarcinomas which include malignancies such as most colon cancers, renal-cell carcinoma, prostate cancer and/or testicular tumours, non-small cell carcinoma of the lung, cancer of the small intestine and cancer of the esophagus. The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. Exemplary carcinomas include those forming from tissue of the cervix, lung, prostate, breast, head (including brain) and neck, colon, skin and ovary. The term “carcinoma” also includes carcinosarcomas, e.g., which include malignant tumours composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation.

Preferably the diseased material is a tumour. The term “tumour” is intended to include tumour cells and the vasculature of the tumour, for example the endothelial cells in the blood vessels of the tumour. The tumour may be benign or malignant. Preferably the tumour is malignant.

As used herein “treatment of cancer” is intended to include the killing of cancer cells or making tumours more sensitive to the effects of radiation and or chemotherapy.

The expression “treatment of a tumour” is intended to include any treatment that destroys or inactivates tumour cells or inhibits or destroys the vasculature of the tumour

A yet further aspect of the invention provides a method of treating the body, body part, tissue, cell, or body fluid of a subject, the method comprising the step of medically imaging the body, body part, tissue, cell or body fluid; administering a cell according to the first aspect of the invention and exposing the cell to a magnetic field. The administration of the cell according to the invention may occur prior to, during or after the medical imaging, or a combination thereof.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

### BRIEF DESCRIPTION OF THE DRAWINGS

FIG. 1: A primary human macrophage (red membrane dye), 7 days post-internalization of 1-2 μm RGD-coated magnetic particles (yellow).

FIG. 2: Transverse section of a T47D spheroid, with a central necrotic area (N) of cell debris, surrounded by two layers of viable cells. The outer layer (blue) consists of normoxic cells, and the inner layer is hypoxic (red staining is for the hypoxic marker, HIF-1α. See arrows).

FIG. 3: Section of a human breast tumour spheroid, showing macrophage accumulation (stained brown, see arrows) in the inner hypoxic rim around the central necrotic area (N).

### MATERIALS AND METHODS

Particle Synthesis—Biocompatible magnetic nanoparticles, primarily composed of a magnetite (Fe₃O₄) and/or maghemite (γFe₂O₃) core with either a silica, dextran, or PVA coating are synthesised following the methods outlined by them in Santra et al. (2001) and Pardoe et al. (2001). Commercially available magnetic micro- and nanoparticles with varying surface chemistry are also used to load the macrophages. The magnetic properties of all particles used in these experiments are characterized with a Quantum Designs MPMS Superconducting Quantum Interference Device (SQUID) magnetometer and these parameters are used as input into the mathematical modelling discussed below.

Coatings and Particle Loading—The polymer coating of the magnetic particles is functionalized and coated with molecules which facilitate membrane adhesion and phagocytosis.

Isolation and Non-Adherent Culture of Human Macrophages—Monocytes are isolated from blood samples taken from healthy volunteers. Blood is collected into sodium citrate (final volume 10%) and layered onto a Ficoll gradient. After centrifugation, the mononuclear cell layer is collected and monocytes purified from this fraction using CD14 magnetic beads as described by us previously in Griffiths et al. (2000). These cells are then cultured in 5% autologous serum/RPMI in non-adherent, sterile Teflon bags for a further 7 days until differentiated into macrophages. Our previous immunohistochemical staining for the pan macrophage marker, CD68, has indicated that >95% of the cells present in these 7 day cultures are monocyte-derived macrophages (MDMs) (Burke et al. 2001 & 2003).

Effects of Magnetic Particle Loading on Macrophages—initial studies on the loading of primary human osteoblasts indicate that even large particles (up to 4.5 μm) are well tolerated by such primary cells for up to 21 days in culture (Cartmell et al., 2002, 2003).

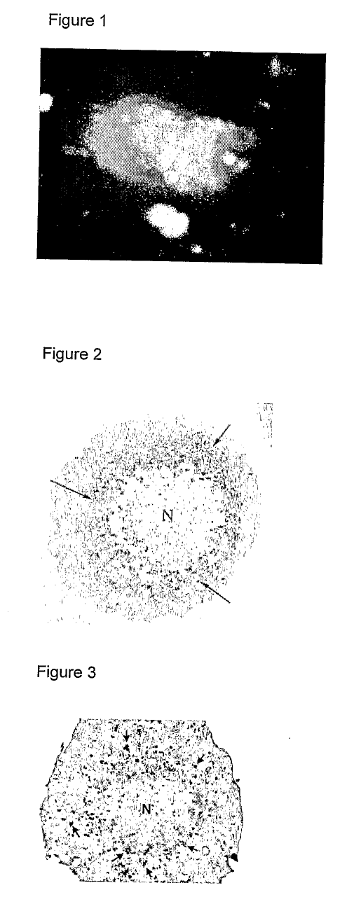

Viability assays are performed and alterations in gene expression are investigated using an Affymetrix gene array. Internalization is using an Olympus Flouview 3D confocal laser scanning microscope and backscattered electron imaging using a HITACHI S-4500 field emission gun scanning electron microscope with a resolution of ˜2 nanometers (FIG. 1).

In Vitro Evaluation of Magnetic Targeting Using Tumour Spheroids

Generation of Human Tumour Spheroids—and Their Infiltration with Human Macrophages—Tumour spheroids are grown in non-adherent, agarose overlay cultures to 800-900 um in diameter from the breast cancer cell line, T47D. This results in spheroids with outer, well-oxygenated cell layers (approx. 200 um wide) and an inner, hypoxic area of viable tumour cells (200-250 um wide) around a central, necrotic area (FIG. 2). We have previously used antibodies to HIF-1 to stain hypoxic tumour cells in the inner hypoxic rim of spheroids (FIG. 2) and shown macrophages to accumulate in these areas following 1-3 days of co-culture with primary MDMs (see FIG. 3). Spheroids are co-cultured with 50,000 primary human macrophages. The total number of macrophages infiltrating spheroids is assessed using flow cytometry (i.e. collagenase dispersal of the spheroids followed by FACS analysis using CD14/CD68 antibodies). The distribution of macrophages inside spheroids is assessed in parallel cultures using immunohistochemistry for CD68 on wax/frozen sections of MTS. This confirms that macrophages are accumulating specifically in the inner, hypoxic areas of spheroids in these co-cultures.

Magnetic Targeting of Spheroids—External rare earth magnets are used to stimulate uptake of magnetic macrophages to the above ‘still’ cultures of spheroids in 96 well plates. These magnets produce high field/gradient products which exert a translational force on the magnetic particles internalized by macrophages. Optimization of the targeting parameters (field strength, gradient, particle size and magnetic properties) is accomplished via mathematical modelling and magnetic field profiles are mapped using a Redcliffe Diagnostics MagScan system. Once this has been done using non-transfected macrophages, macrophages bearing reporter or therapeutic genes are used.

Gene Transfer to Macrophages—Macrophages are infected with a replication defective adenovirus bearing a reporter gene, LacZ under the control of either a constitutively active (CMV) promoter or a hypoxia-regulated response element (HRE). The latter is only expressed when macrophages migrate into the central, hypoxic areas of tumour spheroids (as described previously in Griffith et al. 2000 & Burke et al. 2003). High level gene transfer to macrophages (>85% efficiency in CEL's laboratory) is achieved using such replication-deficient adenoviruses as adv5 (Griffith et al. 2000). As mild hypoxia (3-10% O₂) exists in normal tissues and these would also be infiltrated by transfected macrophages, it is important to use an HRE that is only activated by the extremely low levels of oxygen present in malignant tumours. To this end, a form of HRE (termed an anoxia response element; ARE) only activated in human cells under such pathologically levels of hypoxia/anoxia (<0.1 O₂) has been developed (Ameri et al. 2002 & 2004).

Macrophages are then loaded with biocompatible magnetic nanoparticles and incubated with spheroids in the presence or absence of a high gradient magnetic field (as described above). CMV-driven β-gal expression by macrophages (magnetic vs nonmagnetic) is then quantified in whole, macrophage-infiltrated spheroids using β-GLO FACS analysis—ie. following enzymatic dispersion of cells in spheroids. Alternatively, HRE-driven β-gal expression is visualised in the inner, hypoxic areas of spheroids using x-gal staining of sections of frozen spheroids. If the overall level of uptake of transfected macrophages is increased and/or the level of these cells in the hypoxic, inner core of the spheroid using the ‘nanomagnetic’ approach, the next stage is to replace the reporter gene (LacZ) by a therapeutic gene such as P450 (Griffiths et al. 2000), or the cDNA encoding the potent, new anti-angiogenic peptide, Alphastatin (Staton et al. 2004), and the study repeated to see if the use of magnetic particles also increases the expression of these genes in hypoxic tumour areas.

In Vitro Evaluation of Magnetic Hyperthermia Using Tumour Spheroids

Generation of Spheroid Hyperthermia In Vitro—Once magnetic macrophages have been taken up by spheroids in both the still and flow models described above, a high frequency (˜0.05-1.2 Mhz) electromagnetic field (0-15 kA/m) is applied to via magnetic coil systems. These electromagnetic fields couple to the magnetic particles within the macrophages inducing heat primarily via hysteresis cycling and phase lag of the magnetization vector in superparamagnetic particles according to the equations:

P=μ₀f [symbol] HdM and P_{SPM}=μ₀πfx″H²

where P is the power loss, μₒ is the magnetic permeability of free space, f is the frequency of the magnetic field, H is the magnetic field strength, M is the magnetization of the particle and x″ is the imaginary (phase lag) component of magnetic susceptibility. Heat generated from this process is transferred from the particles to the surrounding cells. Tumour cell and macrophage destruction throughout spheroids (including the inner, hypoxic areas) will be assessed by TUNEL staining of spheroid sections.

Magnetic particle hyperthermia is used in two sets of experiments. In the first case, it is used in combination with the gene therapy technique described above in order to try to destroy the loaded macrophages after the transfection of therapeutic and reporter genes. This prevents the macrophages from promoting cancer growth if they are allowed to remain in the tumour for extended periods of time as previously described. In the second case, this technique is used in a more classical magnetic particle hyperthermia sense, where the objective is not the delivery and transfection of therapeutic genes but rather the destruction of the tumour mass via heating.

Mathematical Modelling

From a modelling standpoint it is helpful to split the process into three stages: (1) magnetic targeting of macrophages to the tumour region; (2) their accumulation in hypoxic regions of the tumour; (3) magnetic hyperthermia. The modelling issues, in each of these areas, are outlined below.

(1) Macrophages are loaded with magnetic particles and introduced into an artery feeding the twnour region. A steady magnetic field, with maximum intensity close to the tumour, is used to pull the macrophages out of suspension and onto the endothelial walls. The main aim of the modelling at this stage is to predict the types of vessel, and the positions of vessels, in which the macrophages come out of suspension. The modelling involves balancing Stokes' drag (arising from the interaction of macrophage with the blood flow in the vessel) with magnetic force on a macrophage. Outcomes of the modelling include the design of magnetic fields (strengths and intensity distribution) and the level of magnetic particle doping necessary to optimise the distribution of macrophages over the vasculature of the tumour region.

(2) Once a macrophage reaches the endothelial wall it binds to it via ligands, expresses chemicals that weaken the vessel wall letting it through into the inter-cellular space. It then permeates into the inter-cellular space and is attracted to regions of low oxygen concentration via chemotaxis. This stage is modelled with a phase model which describes the viscoelastic interactions of extracellular matrix, macrophages/macrophages, tumour cells and water. The modelling challenges are to incorporate the chemotactic ‘force’ acting on the macrophages/macrophages into the multiphase flow framework and to account for differentiation of macrophages into macrophages. A key issue to address with the aid of this model, and one that is important to the success of this therapy, is to what extent are doped macrophages/macrophages and undoped macrophages/macrophages competing for space within the tumour (3) Once the magnetically doped macrophages have reached their target a localised oscillating magnetic field is applied to the tumour. The magnetisation of the magnetic particles within the macrophages undergo hysteresis, with the oscillations of the field, and release thermal energy as they do so. The release of energy causes localised heating which is used to destroy tumour cells. This heating is modelled by a diffusion equation for the temperature with sources terms at the positions of macrophages (and hence magnetic particles). The strength of these source terms is estimated by exploiting the magnetic hysteresis curve for magnetite. The model is used to calculate the strength of magnetic field that should be applied to the tumour to give the desired heating effects.

### EXAMPLE

RGD-coated biocompatible magnetic microparticles (1-2 μm in size), consisting of a magnetite (Fe₃O₄) and maghemite (γFe₂O₃) core were prepared in using methods similar to those outlined previously by them (Cartmell et al., 2002, 2003; Hughes et al., 2003). Blood monocytes were isolated from human blood and allowed to differentiate in culture into macrophages for 8 days. The cells membranes were then labelled with the red fluorescent cell tracker dye PHK-26 (Sigma) (using the manufacturers guidelines), and incubated with the magnetic microparticles (25 μg/ml). Cells were incubated with the particles overnight to allow their phagocytosis. Unbound particles were then removed by PBS washes, before cells were maintained under routine culture conditions for up to 7 days. Laser scanning confocal microscopy was then used in conjunction with image analysis software (Olympus, Fluoview) to track the location of particles (yellow) in relation to macrophage cell membranes (red). Cells were imaged over a period of 1-7 days post ingestion of particles to determine whether the particles remained internalized. FIG. 1 shows a macrophage seven days after ingestion of magnetic particles (assessed using vertical section imaging). This pilot study confirmed both the rapid uptake of these particles by macrophages (particles were completely internalized at the one-day analysis time-point) and their retention within cells for at least seven days. In this pilot work, microparticles rather than nanoparticles were used in order to accurately visualize the three-dimensional distribution of the particles within the cells using confocal microscopy. This data is in agreement with our other studies which show rapid uptake of RGD-coated magnetic micro- and nanoparticles by primary human osteoblasts and bone marrow stromal cells (Cartmell et al., 2002 & 2003).

### REFERENCES

Ameri K, Burke B, Lewis C E & Harris A L (2002). Regulation of a rat VL30 element in human breast cancer cells in hypoxia and anoxia: role of HIF-1. Brit. J Cancer 87: 1173-81.
Ameri K, Lewis C E, Raida M, Hai T & Harris Al (2004). Anoxic induction of ATF-4 in human cancer cells via HIF-1 independent pathways of protein stabilisation. Blood (in press).
Andreesen R, Hennemann B & Krause S W. (1998). Adoptive immunotherapy of cancer using monocyte-derived macrophages: rationale, current status, and perspectives. J Leuk Biol. 64:419-26.
Baltholeyns J & Lopez M. (1994) Immune control of neoplasia by adoptive transfer of macrophages: potentiality for antigen presentation and gene transfer. Anticancer Res. 14, 2673-2676.
Bartholeyns J, Romet-Lemonne J L, Chokri M & Lopez, M. (1996) Immune therapy with macrophages: present status and critical requirements for implementation. Immunobiology 195: 550-562.
Burke B, Tang N, Corlce K P, Tazzyman D, Ameri K, Wells M & Lewis C E. (2002a). Expression of HIF-1 alpha by human macrophages: implications for the use of macrophages in hypoxia-regulated cancer gene therapy. J Path. 196:204-212.
Burke B, Sumner S, Maitland N & C E Lewis (2002b). Macrophages in gene therapy: cellular delivery vehicles and in vivo targets. J. Leuk. Biol. 72: 417-428.
Burke B, Gouinnidis A, Corke K P, Gill D, Wells M & Lewis C E. (2003). Hypoxia-induced gene expression in human macrophages: implications for ischemic tissues and hypoxia-regulated gene therapy. Am. J Pathol. 163. 1233-42.
Burton J L, Wells J M, Corke K P, Maitland N, Hamdy F C & Lewis C E. (2001). Macrophages accumulate in avascular, hypoxic areas of prostate tumours: implications for the targeted therapeutic gene delivery to such sites. J Pathol 192: 8A.
Brown J M & Giaccia A J. (1998). The unique physiology of solid tumours: opportunities (and problems) for cancer therapy. Cancer Res. 58: 1408-16.
Brown J M. (2000). Exploiting the hypoxic cancer cell: mechanisms and therapeutic strategies. Mol Med Today 6:157-62.
Cartmell, S H, J Dobson, S Verschueren, S Hughes, A J El Haj (2002) Mechanical conditioning of bone cells in vitro using magnetic microparticle technology. European Cells & Materials, 4 (Supp. 2): 42-44.
Cartmell, S H, J Dobson, S Verschueren, A El Haj (2003) Preliminary analysis of magnetic particle techniques for activating mechanotransduction in bone cells. IEEE Trans. NanoBiosci. 1: 92-97.
Chokri M, Lallot C, Ebert M, Poindron P & Batholeyns, J. (1990). Biodistribution of indium-labelled macrophages in mice bearing solid tumors. Int. J. Immunol. 1: 79-84.
Goodwin, S C Peterson, C L Hoh, C Bittner, C A (1999) Targeting and retention of magnetic targeted carriers (MTC) enhancing intra-arterial chemotherapy. J. Magn. Magn. Mater. 194:132-139.
Griffiths L, Binley K, Iqball S, Kan O, Maxwell P, Ratcliffe P, Lewis C E, Harris A L, Kingsman S & Naylor S M. (2000). The macrophage: a novel system to deliver gene therapy to pathological hypoxia. Gene Therapy 7:255-62.
Hughes, S, J Dobson, A J El Haj (2003) Mechanical stimulation of calcium signaling pathways in human bone cells using ferromagnetic microparticles: Implications for tissue engineering. Eur. Cells & Mater. 6 (Supp. 2): 43.
Leek R D, Lewis C E, Whitehouse R, Greenall M, Clarke J & Harris A L. (1996). Association of macrophage infiltration with angiogenesis and prognosis in invasive breast carcinoma. Cancer Res 56: 4625-9.
Lewis J S, Landers R, Underwood J C E, Harris A L & Lewis C E. (2000). Expression of VEGF by macrophages is upregulated in avascular areas of breast carcinomas. J Pathol. 192: 150-58.
Lewis J S, Lee J, Underwood J C E, Harris A L & Lewis C E. (1999). Macrophage responses to hypoxia: implications for disease mechanisms. J Leuk Biol 66:889-900.
Lübbe, A S and 17 others (1996) Clinical experiences with magnetic drug targeting: a phase I study with 4′-epidoxorubicin in 14 patients with advanced solid tumors. Cancer Res. 56: 4686-4693.
Kouraklis G. (2000). Gene therapy for cancer: from the laboratory to the patient. Dig Dis Sci. 45:1045-52.
Murdoch C, Gouinnidis A & Lewis C E. (2004). Mechnanism regulating the recruitment of macrophages into human tumours: accumulation in hypoxic/necotic areas. Blood (submitted; invited review).
Mosbach, K & Schröder, U (1979) Preparation and application of magnetic polymers for targeting of drugs. FEBS Lett. 102: 112-116.
Pankchurst, Q A, J Connoly, S K Jones & J Dobson (2003) The underlying physics of biomedical applications of magnetic nanoparticles. J. Phys. D. 36: R167-R181.
Pardoe, H, W Chua-anusorn, T G St. Pierre, J Dobson (2001) Structural and magnetic properties of nanoscale magnetic particles synthesised by coprecipitation of iron oxide in the presence of dextran or polyvinyl alcohol. J. Magn. Magn. Mat. 225: 41-46.
Pulfer S K, Gallo J M (1998) Enhanced brain tumor selectivity of cationic magnetic polysaccharide microspheres. J. Drug. Target. 6: 215-227.
Santra S, Tapec R, Theodoropoulou N, Dobson J, Hebard A & W Tan (2001) Synthesis and characterization of silica-coated iron oxide nanorparticles in microemulsion: the effect of non-ionic surfactants. Langmuir 17: 2900-2906.
Senyie A, Widder K & Czerlinski C (1978) Magnetic guidance of drug carrying microspheres. J. Appl. Phys. 49: 3578-3583.
Staton C A, Brown N J, Rodgers G R, Corke K P, Tazzyman S, Underwood J C E & Lewis C E. (2004). Alphastatin, a 24 amino acid fragment of human fibrinogen, is a potent new inhibitor of angiogenesis. Blood (in press).
Widder, K J, Senyei, A E & Scarpelli, D G (1978) Magnetic microspheres: a model system for site specific drug delivery in vivo. Proc. Soc. Exp. Biol. Med. 58: 141-146.
Vaupel P, Kallinowski F & Okunieff P. (1989). Blood flow, oxygen and nutrient supply, and metabolic microenvironment of human tumours: a review. Cancer Res. 49: 6449-65.
Vile R G, Russell S J & Lemoine N R. (2000). Cancer gene therapy: hard lessons and new courses. Gene Ther. 7(1):2-8.

1. A monocyte, or monocyte-derived cell, comprising a magnetic material.

2. A monocyte, or monocyte-derived cell, as claimed in claim 1 which is a macrophage.

3. A monocyte, or monocyte-derived cell, as claimed in claim 1 wherein the magnetic material is a magnetic particle.

4. A monocyte, or monocyte-derived cell, as claimed in claim 3 wherein the magnetic material is a nano-particle.

5. A monocyte, or monocyte-derived cell, as claimed in claim 1 wherein the magnetic material is a ferrofluid.

6. A monocyte, or monocyte-derived cell, as claimed in claim 1 wherein the magnetic material comprises a biocompatible coating.

7. A monocyte, or monocyte-derived cell, as claimed in claim 1 wherein the magnetic material comprises a biocompatible coating wherein the biocompatible coating is a metal.

8. A monocyte, or monocyte-derived cell, as claimed in claim 1 wherein the magnetic material comprises a biocompatible coating wherein the biocompatible coating is a polymer.

9. A monocyte, or monocyte-derived cell, as claimed in claim 1 wherein the magnetic material comprises a biocompatible coating which is a polymer and wherein the polymer is selected from the group consisting of dextran, polyvinyl alcohol (PVA), polyethylenimine (PEI), or silica.

10. A monocyte, or monocyte-derived cell, as claimed in claim 1 wherein the magnetic material comprises a biocompatible coating wherein the biocompatible coating is provided with a biological molecule which may function to facilitate adhesion of the magnetic particle to the monocyte, or monocyte derived cell.

11. A monocyte, or monocyte-derived cell, as claimed in claim 10 wherein the biological molecule is selected from the group consisting of RGD, transferrin, collagen, fibronectin/fibrin, ion channel receptors, cell specific integrin receptors or any cell surface antigen.

12. A monocyte, or monocyte-derived cell, as claimed in claim 1 which comprises a therapeutic agent.

13. A monocyte, or monocyte-derived cell, as claimed in claim 12 wherein the therapeutic agent is selected from the group consisting of a chemotherapeutic agent, a radiotherapeutic agent, a therapeutic gene, or part thereof, or a protein or part thereof.

14. A monocyte, or monocyte-derived cell, as claimed in claim 12 wherein the therapeutic agent is selected from the group consisting of DNA, RNA, interfering RNA (RNAi), a peptide, polypeptide, an antibody, an antibody fragment or an aptamer

15. A monocyte, or monocyte-derived cell, as claimed in claim 1 which comprises a responsive element wherein the responsive element is a gene or part thereof that is responsive to conditions in or around a tumour.

16. A monocyte, or monocyte-derived cell, as claimed in claim 15 wherein expression of the responsive element is regulated by anoxia.

17. A monocyte, or monocyte-derived cell, as claimed in claim 15 wherein expression of the responsive element is regulated by hypoxia.

18. A monocyte, or monocyte-derived cell, as claimed in claim 15 wherein the responsive element is operably linked to a therapeutic gene so as to regulate expression of the therapeutic gene.

19. A monocyte, or monocyte-derived cell, as claimed in claim 15 wherein the responsive element comprises all or part of a therapeutic gene.

20. monocyte, or monocyte-derived cell, as claimed in claim 15 wherein the responsive element consists of all or part of a therapeutic gene.

21. A monocyte, or monocyte-derived cell, as claimed in claim 15 wherein the responsive element is selected from the group consisting of a tumour suppressor gene, antigenic gene, cytotoxic gene, cytostatic gene, cytokine gene, chemokine gene, pharmaceutical protein gene, pro-apoptotic gene, pro-drug activating gene and an anti-angiogenic gene.

22. (canceled)

23. A pharmaceutical composition comprising a monocyte, or monocyte-derived cell, as claimed in claim 1 and a pharmaceutically acceptable carrier or diluent.

24. A method of targeting a magnetic material to a diseased material in a subject, the method comprising the administration of a monocyte, or monocyte-derived cell, as claimed in claim 15 to the subject's body, body part, tissue or body fluid.

25. A method for directing a monocyte, or monocyte-derived cell, as claimed in claim 1 to a diseased material in a subject the method comprising administering the monocyte, or monocyte derived cell, to the subject's body, body part, tissue or body fluid and exposing said monocyte, or monocyte derived cell, to a magnetic field.

26. A method as claimed in claim 24 wherein the method is carried out in vivo.

27. A method as claimed in claim 24 wherein the method is carried out ex vivo.

28. A targeted thermotherapy system for treating diseased material in a subject, the system comprising:
i) a monocyte, or monocyte derived cell, as claimed in claim 1; and
ii) a magnetic energy source to heat the magnetic material within the cell.

29. A method of treating a diseased material in a subject the method comprising the administration of a monocyte, or monocyte derived cell, as claimed in claim 15, to a subject's body, body part, tissue or body fluid.

30. A method as claimed in claim 29 wherein the method is gene therapy.

31. A method of treating a diseased material in a subject the method comprising the administration of a monocyte, or monocyte derived cell, as claimed claim 1 to a subject's body, body part, tissue or body fluid and the administration of a magnetic energy source to destroy, rupture or inactivate the diseased material.

32. A method as claimed in claim 29 wherein the treatment is hyperthermia.

33. (canceled)

34. (canceled)

35. A method of treating the body, body part, tissue, cell, or body fluid of a subject, the method comprising the step of medically imaging the body, body part, tissue, cell or body fluid; administering a monocyte, or monocyte derived cell, as claimed in claim 1 and exposing said monocyte or monocyte derived cell to a magnetic field.

36. A method as claimed in claim 25 wherein the method is carried out in vivo.

37. A method as claimed in claim 25 wherein the method is carried out ex vivo.
